(19) United States
(12) Patent Application Publication
(10) Pub. No.: US 2023/0302231 A1
(43) Pub. Date: Sep. 28, 2023

(54) Needle Assembly and Method

(71) Applicant: Kevin Michels, Spokane, WA (US)

(72) Inventor: Kevin Michels, Spokane, WA (US)

(21) Appl. No.: 17/705,214

(22) Filed: Mar. 25, 2022

(51) Int. Cl. A61M 5/32; A61M 5/34

(52) U.S. Cl. CPC A61M 5/3276; A61M 5/3213; A61M 5/347; A61M 2005/3208; A61M 2205/586; A61M 2205/583; A61M 2205/582

(57) ABSTRACT

A needle assembly is provided having a needle cannula, a hub, and a pair of opposed radially outwardly extending paddles. The needle cannula has a proximal end and a distal end with a puncture tip. The hub has a proximal end and a distal end. The hub supports the needle cannula at a distal end. A twist fitting connector is provided at the proximal end. The pair of opposed radially outwardly extending paddles each have a digit pad configured on the hub in an anatomically preferential position when the needle is affixed to a syringe with the connector. In use, a user palm-grasps a barrel of the syringe so as to point the puncture tip away from a user when receiving at least one digit on at least one of the digit pads during mating and demating from the syringe. A method is also provided.

### TECHNICAL FIELD

Examples disclosed herein are generally related to the field of male and female joining devices, and more particularly related to needles, caps and needle/cap assemblies, including hypodermic needles and caps.

### BACKGROUND

It was previously known to use male and female connectors and caps to mate and demate (i.e. screw and unscrew the needles from the syringe) hollow safety needles from a syringe or tubular body for injecting or withdrawing fluids relative to a body or a wound. Recent designs incorporate small needle hubs that are mated within a receiving circumferential groove within a syringe body. However, there exist problems when mating and demating such a needle from a syringe. Such problems have recently been exacerbated by pandemic conditions that have strained personal protection equipment resourcing and quality, leading to greater variations in elasticity, conformity of materials and thickness, and dexterity in medical and surgical gloves. Gloved hands are a necessity when using such needle assemblies in exposure with bodily fluids. Improvements are needed to facilitate safe mating, demating, and use of needle assemblies so as to reduce or eliminate risk of inadvertent needle punctures through a user's gloves. Further improvements are needed to reduce exposure to two-handed removal when removing a loaded and biologically contaminated used needle from a syringe. Furthermore, as more medications are used with home injections and infusions, the need for simpler removal of the needle will be required. Additionally, as the work force ages, the need for health care workers to be able to mate and demate needles will be more critical as older workers will have a higher incidence of arthritis and neuropathy. Lastly, medications are becoming more expensive and, as patients are starting to have to infuse them at home, being able to unscrew draw up needles from syringes to place smaller injection needles onto a syringe will be critical. The older age individuals will have decreased grip strength and increased incidence of osteoarthritis, rheumatoid arthritis and neuropathies from diseases like diabetes which will make manipulation of the needles more problematic.

### SUMMARY

An apparatus and method are provided for mating and demating a needle from a fluid supply chamber, such as a syringe, and for mating a needle and cap assembly onto a syringe and for removing a needle in a mated position relative to a syringe in both a manner that reduces risk of inadvertent needle puncture.

According to one aspect, a needle assembly is provided having a needle cannula, a hub, and a pair of opposed radially outwardly extending paddles. The needle cannula has a proximal end and a distal end with a puncture tip. The hub has a proximal end and a distal end. The hub supports the needle cannula at a distal end. A twist fitting connector is provided at the proximal end. The pair of opposed radially outwardly extending paddles each have a digit pad configured on the hub in an anatomically preferential position when the needle is affixed to a syringe with the connector. In use, a user palm-grasps a barrel of the syringe so as to point the puncture tip away from a user when receiving at least one digit on at least one of the finger pads during mating and demating from the syringe.

According to another aspect, a needle assembly is provided having a needle cannula, a hub, a twist fitting connector, and a radially outwardly extending lever arm. The needle cannula has a proximal end and a distal end with a puncture tip. The hub has a proximal end and a distal end. The hub supports the needle cannula at a distal end. The twist fitting connector is provided at the proximal end. The radially outwardly extending lever arm has a digit pad configured on the hub in an anatomically preferential position when the needle is affixed to a syringe with the connector. Accordingly, a user is induced to palm-grasp a barrel of the syringe in a manner that points the puncture tip away from a user when receiving a digit on the finger pad during mating and demating from the syringe.

According to yet another aspect, a method is provided for demating a needle from a syringe. The method includes: providing a needle assembly having a cannula, a hub, a twist fitting connector on the hub configured to affix the hub onto a syringe, and at least one radially outwardly extending lever arm on the hub having a digit pad affixed onto a syringe; and while affixed onto the syringe, pointing the cannula away from a user; engaging a user's digit with the digit pad; and pivotally urging the digit pad with the user's digit to rotate and decouple the twist fitting connector and remove the needle assembly from the syringe.

### BRIEF DESCRIPTION OF THE DRAWINGS

FIG. 1 is a perspective view from above of a prior art needle and syringe.

FIG. 2 is a perspective view of the prior art needle and syringe illustrating an anatomic needle puncture risk when mating/demating the needle from the syringe.

FIG. 3 is a perspective view of the prior art needle and syringe showing a discovered glove-locked condition of a gloved finger overwrapped and entrapped onto a hub of the needle likely to generate a risk of an inadvertent needle jab to a user.

FIG. 4 is an enlarged perspective view from the encircled region 4 of FIG. 3 showing the overwrapped and entrapped glove and needle.

FIG. 5 is a simplified perspective view showing a demonstrative strip of elastic nitrile glove engaging and beginning to overwrap with a prior art needle hub.

FIG. 6 is a simplified perspective view later in time and showing the demonstrative elastic strip wrapped further around the needle hub than depicted in FIG. 5 to form an entrapped overwrap of the elastic strip.

FIG. 7 is a simplified perspective view of the entrapped and overwrapped elastic strip stretched as a user moves their entrapped hand away from the needle causing a loaded condition that could lead to an inadvertent needle puncture.

FIG. 8 is a simplified perspective view of a user's gloved hands and a needle and syringe after the stretched, entrapped and overwrapped elastic segment of glove launches a needle and syringe from a user's right hand into a palm of a user's left hand.

FIG. 9 is a simplified perspective view of a user's gloved hands with a needle and syringe and showing a user grasping a needle hub along a first index finger joint and thumb during mating and/or demating of the needle from the syringe using a traditional method for removal of a syringe.

FIG. 10 is a simplified perspective view of a user's gloved hands with a needle and syringe and showing a user grasping a needle hub along a second index finger joint and thumb during mating and/or demating of the needle from the syringe.

FIG. 11 is a perspective view of a prior art needle and syringe assembly with a mating/demating complementary cap and showing a misalignment needle puncture risk when mating cap 72 onto needle 12.

FIG. 12 is a side view taken along plane 12 of FIG. 11 showing the needle punctured through the cap resulting from cap misalignment.

FIG. 13 is a perspective needle end view from above of one overwrap-preventing needle with a complementary syringe and further showing two alternative variation overwrap-preventing needles.

FIG. 14 is a perspective view of a user mating the one overwrap-preventing needle and cap of FIG. 13 onto a syringe;

FIG. 15 is a perspective view of a user mating the one overwrap-preventing needle of FIG. 13 onto a syringe without a cap and with the needle pointed away from a user;

FIG. 16 is a perspective view of a user demating the one overwrap-preventing needle of FIG. 13 from a syringe in a two-handed manner that reduces the risk of puncture by having the needle pointed away from the user by changing the biomechanics of how the needle is easily manipulated and removed.

FIG. 17 is a perspective view of a user demating the one overwrap-preventing needle of FIG. 13 from a syringe in a single-handed manner that reduces the risk of puncture by having the needle pointed away from the user.

FIG. 18 is a front elevational view of the one overwrap-preventing needle and syringe of FIGS. 13-17.

FIG. 19 is a right side view of the one overwrap-preventing needle and syringe of FIGS. 13-18.

FIG. 20 is a top end view of the one overwrap-preventing needle and syringe of FIGS. 13-19.

FIG. 21 is a vertical sectional view of the one overwrap-preventing needle and syringe of FIG. 13 in assembly taken along line 21-21 of FIG. 20.

FIG. 22 is a needle end perspective view from above of the one overwrap-preventing needle of FIGS. 13-21.

FIG. 23 is a connector end view of the one overwrap-preventing needle of FIG. 22.

FIG. 24 is a front elevational view with the needle pointed down of the one overwrap-preventing needle of FIGS. 13-23.

FIG. 25 is a right side view of the needle of FIG. 24.

FIG. 26 is a needle end view of the one overwrap-preventing needle of FIGS. 19-25.

FIG. 27 is a perspective view from a closed end of a cap for the overwrap-preventing needle of FIGS. 13-26.

FIG. 28 is a top end view of the end cap of FIG. 27.

FIG. 29 is a front elevational view of the end cap of FIG. 28.

FIG. 30 is a right side elevational view of the end cap of FIG. 29.

FIG. 31 is a bottom end view of the end cap of FIG. 29.

FIG. 32 is a front elevational view of an alternative variation overwrap-preventing needle similar to that depicted in FIGS. 13-23, but having a curved leading edge delta configuration and a tactilely detectible surface feature, or input on a removal leverage surface of each tab.

FIG. 33 is a connector end view of the one overwrap-preventing needle of FIG. 32.

FIG. 34 is a connector end perspective view from above of the one overwrap-preventing needle of FIGS. 32-33.

FIG. 35 is an enlarged view of the curved leading edge delta configuration for the tabs taken from the encircled region 35 of FIG. 34.

FIG. 36 is an enlarged view corresponding with that shown in FIG. 35, but showing a first alternative curved leading edge delta configuration for one tab having a round raised button.

FIG. 37 is an enlarged view corresponding with that shown in FIG. 35, but showing a second alternative curved leading edge delta configuration for one tab having a round raised button within a cylindrical button-shaped depression.

FIG. 38 is an enlarged view corresponding with that shown in FIG. 35, but showing a third alternative curved leading edge delta configuration for one tab having a linear array of three raised delta darts.

FIG. 39 is a table illustrating biomechanics factors for various needle designs.

### DETAILED DESCRIPTION OF THE ILLUSTRATED EMBODIMENTS

This disclosure of the invention is submitted in furtherance of the constitutional purposes of the U.S. Patent Laws “to promote the progress of science and useful arts” (Article 1, Section 8).

During healthcare delivery it is typical that needles need to be screwed on and then off when drawing up medications of any type. A large bore needle, usually an 18 gauge needle, is typically used to draw up a medication. In contrast, it usually proves difficult to draw up medications through a small bore needle. If the medication is to be injected directly into the patient, a smaller bore needle, usually a 25, 27, or 30 gauge needle is screwed onto a syringe. The larger needle needs to be unscrewed and removed from the syringe. When on the syringe, the needle needs to be screwed onto the syringe with the threads tight enough that it will provide an airtight seal between the needle hub and the syringe threads. If it is not air tight, the medication will typically either run out of the syringe losing potentially an expensive drug creating an unclean/non-sterile environment or not draw up the medication from the bottle into the syringe because an air tight system is needed to effect the draw. The needle is then inserted into the medicine bottle and the medication is drawn into the syringe. The large needle now needs to be removed from the syringe to then place a smaller needle on to the syringe so the medication can be directly injected into a patient. This is where the proposed present embodiments for the new needle and cap presented herein is beneficial.

When in an operating room and the needle is used in a patient on a sterile operating room field, the needle has to be removed in order to fill up the syringe with more medication (i.e. a local anesthetic). This needle is a “dirty” needle, i.e. has been used on a patient. You cannot easily pull up medications through a small bore needle, as described above. This is critical because the staff are working with a “dirty” needle and it will be continually used through the surgery. There is a reason for reuse, namely, multiple new needles are not typically opened onto an operating room table because each new needle must be counted and accounted for at the end of the case in order to make sure that none have been left in the patient prior to leaving the operating room. Opening more needles to be kept track of on a sterile field increases the risk to the patient. Therefore, it becomes necessary that needles have to be removed from a syringe repeatedly in order to allow for injection of medications or local anesthetic and using a needle only once is not practical. In a clinic nor non-operating room situation, the same restrictions apply where the removal of needles is necessary after medication has been drawn and avoiding contamination of the medication is important.

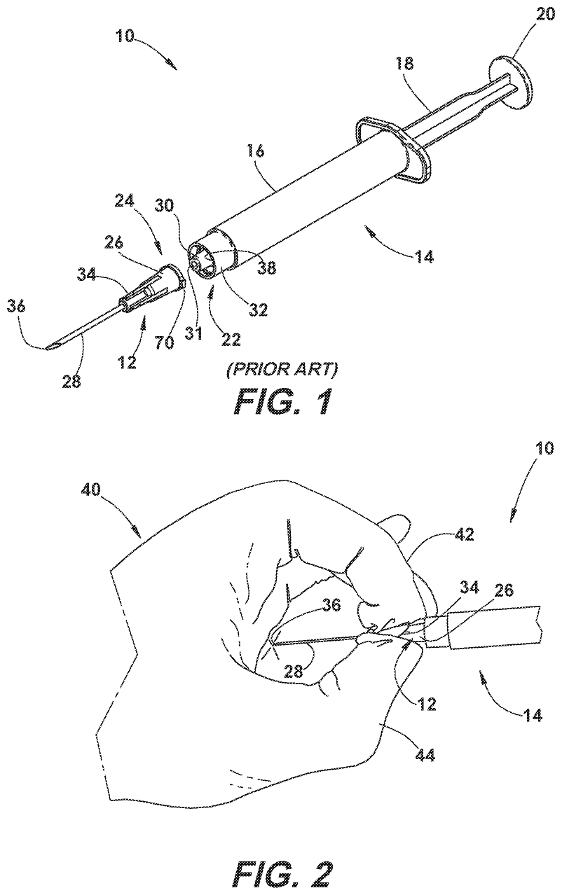

FIG. 1 is a perspective view from above of a prior art needle and syringe assembly 10 including a removable hypodermic needle 12 and a hypodermic syringe 14. Syringe 14 includes a tubular, or cylindrical hollow body 16, a plunger shaft 18, and a plunger end plate 20, wherein a sliding seal end (not shown) on plunger shaft 18 reciprocates in sealing engagement within an inner wall (not shown) of body 16. Needle 12 includes a plastic needle base, or luer lock base 26 affixed about a proximal end of a hollow needle body 28 terminating in a beveled, sharp distal end tip 36. Base 26 includes a circumferentially spaced-apart array of longitudinally extending ribs 34 about a distal end of base 26 configured to facilitate user tactile engagement when mating and demating needle 12 from syringe 14 via a female connector, or male luer lock 22 and complementary male connector, or female luer lock 24. Mating luer locks 24 and 26 each form a portion of a pair of mating and sealing twist fitting connectors. Connector 22 comprises a tapered inner female luer connector 38 formed in an inner surface of an outer hub 32 and an inner frustoconical hub 30 provided coaxially within the surrounding cylindrical outer hub 32. Tapered hub 30 mates in axial tapering engagement with a complementary frustoconical tapered bore (not shown) in hub 26 to form a fluid seal. Male luer lock tabs 70 on needle hub 26 mate in helical engagement with female connector 38. A central bore 31 in a distal end of hub 30 provides fluid transfer between syringe 14 and needle 12.

FIG. 2 is a perspective view of the prior art needle and syringe assembly 10 of FIG. 1 and illustrating a discovered anatomic needle puncture risk when mating/demating the needle 12 from the syringe 14. More particularly, a user is shown grasping hub 26 of needle 12 with a gloved left hand 40 while holding syringe 14 with a right hand (not shown). In order to engage ribs 34 on hub 26 when mating and demating needle 12 from syringe 14, a user articulates gloved hand 40 with index finger, or digit 42 and thumb 44 in opposition on opposed sides of hub 26 in engagement with ribs 34 while taking care to provide clearance between sharp tip 36 on needle body 28 relative to a palm on hand 40.

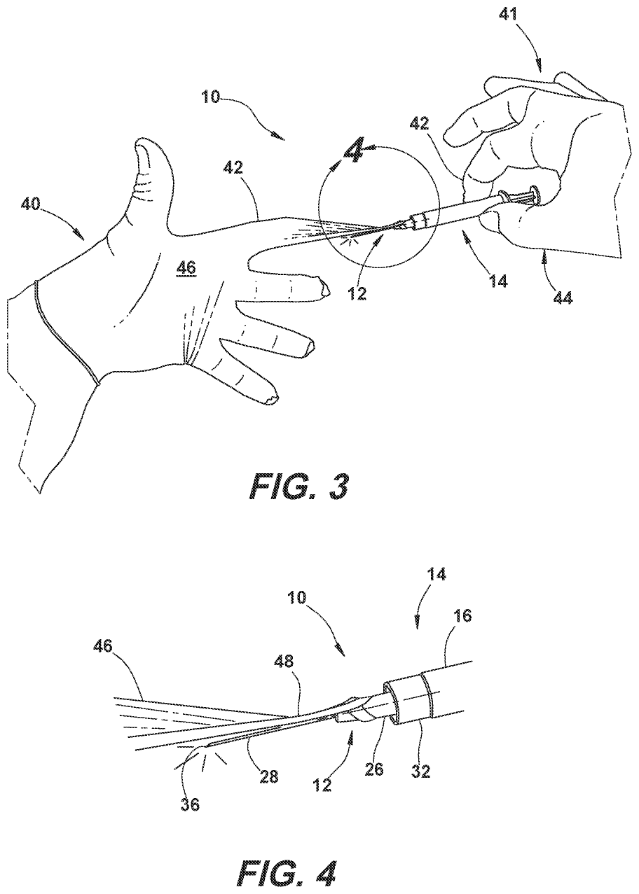

FIG. 3 is a perspective view of the prior art needle and syringe assembly 10 of FIGS. 1-2 showing a recently discovered glove-locked condition of a glove index finger, or digit 42 on glove 46 worn on a user's left hand 40. The glove finger 42 is overwrapped and entrapped onto a hub of needle 12 likely to generate a risk of an inadvertent needle jab or needle stick to a user's left hand 40. Syringe 14 is held between index finger 42 and thumb 44 of a user's right hand 41. A stretched portion, or segment 48 of glove 46 is shown locked in overwrap over hub 26 on needle '12.

FIG. 4 is an enlarged perspective view from the encircled region 4 of FIG. 3 showing the overwrapped and entrapped glove 46 and needle 12 of the prior art needle and syringe assembly 10. It has been discovered that, under certain conditions where glove material and fit are just right, surface features of hub 26 on body 16 adjacent to outer hub 32 of syringe 14 can overwrap and lock strip 48 onto hub 26. Once locked, strip 48 can stretch like a rubber band and cause a risk that tip 36 on needle body 28 can be launched into a user's hand, penetrating glove 46.

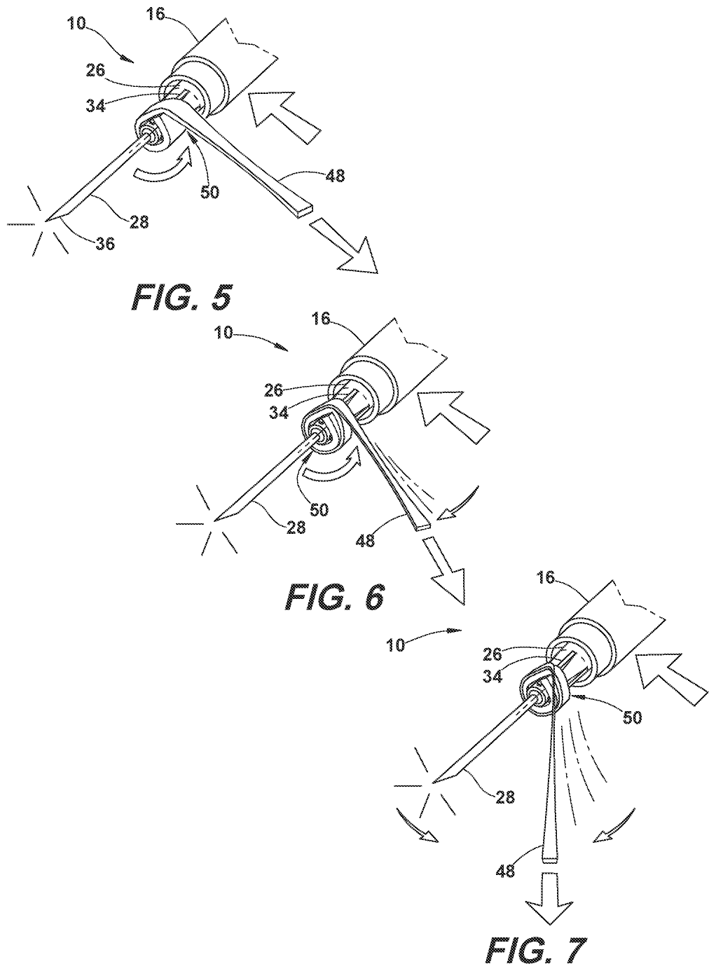

FIG. 5 is a simplified perspective view showing a demonstrative strip 48 of elastic nitrile glove engaging and beginning to overwrap with a prior art needle hub 26 affixed to a body 16 of a needle and syringe assembly 10. More particularly, an axially extending array of circumferentially spaced apart ribs 34 on hub 26 have been found to catch and grip loose portions of a glove, such as strip 48 and wrap around hub 26. A winding 50 of strip 48 begins to form as a complete wrap onto itself that entraps strip 48 onto hub 26.

FIG. 6 is a simplified perspective view later in time and showing the demonstrative elastic strip 48 wrapped further around the needle hub 26 than depicted in FIG. 5 to form an entrapped overwrap, or winding 50 of the elastic strip 26 that locks onto ribs 34 of hub 26. As needle and syringe assembly 10 is moved away by a user grasping body 16 (not shown), strip 48 stretches which further self-locks strip 48 onto hub 26 and begins to provide a potential sling shot launching force to needle body 28.

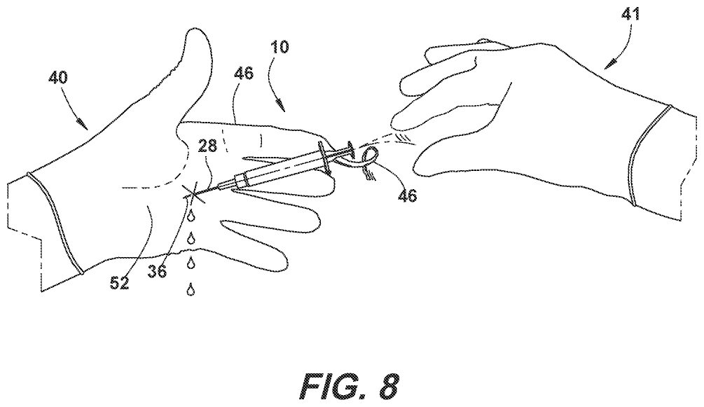

FIG. 7 is a simplified perspective view of the entrapped and overwrapped elastic strip 46 from glove 40 on finger 42 is stretched and rotated relative to needle and syringe assembly 10 as a user moves their entrapped hand away from the needle causing a loaded and stretched condition on strip 48 that could lead to an inadvertent needle puncture 36 into a palm 52 as needle 28 can be driven into a user's hand when body 16 slips from a user's opposing hand (see FIG. 8).

FIG. 8 is a simplified perspective view of a user's left and right hands inside gloves 40 and 41 and a needle and syringe assembly 10 after the stretched, entrapped and overwrapped elastic segment 46 of glove 40 launches a needle 28 of a needle and syringe assembly 10 from a user's right hand into a palm 52 of a user's left hand.

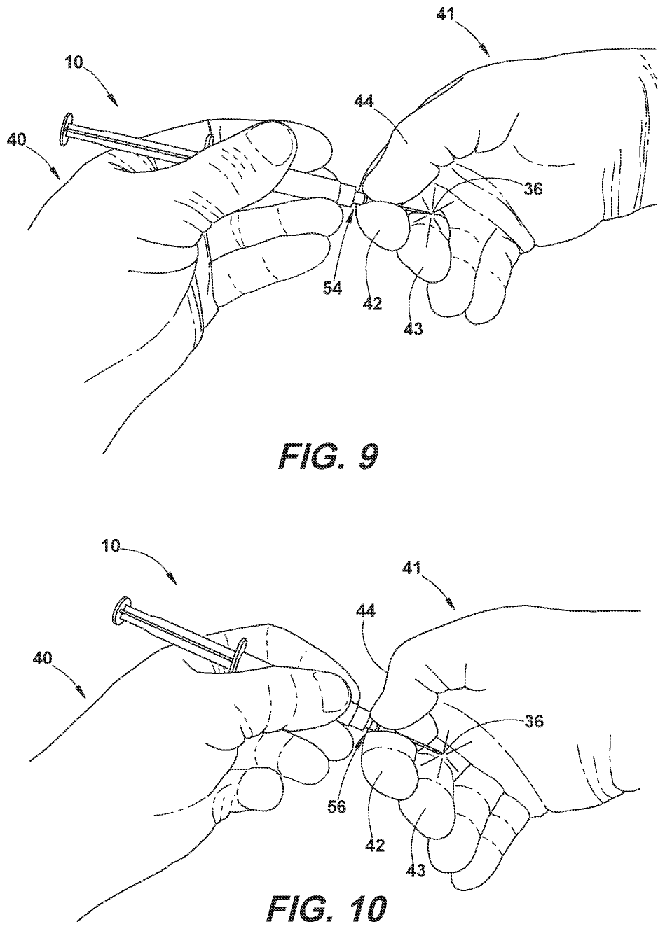

FIG. 9 is a simplified perspective view of illustrating how uncapped needles are traditionally removed with a user's left and right hands in gloves 40 and 41 with a needle and syringe assembly 10 and showing a user grasping a needle hub 26 along a first index finger, or forefinger 42 at first joint 54 and against an opposing thumb 44 during mating and/or demating of the needle from the syringe of assembly 10. Needle 36 lies across joint 54 on index finger 42 and presents a puncture risk with finger 43.

FIG. 10 is a simplified perspective view of how with a traditional removal of a needle when more force is needed a user's left and right hands in gloves 40 and 41 with a needle and syringe assembly 10 and showing a user grasping a needle hub 26 along an index finger, forefinger 42 at a second joint 56 and against an opposing thumb 44 during mating and/or demating of the needle from the syringe of assembly 10. Needle 36 lies across joint 56 on index finger 42 and presents a puncture risk with finger 43.

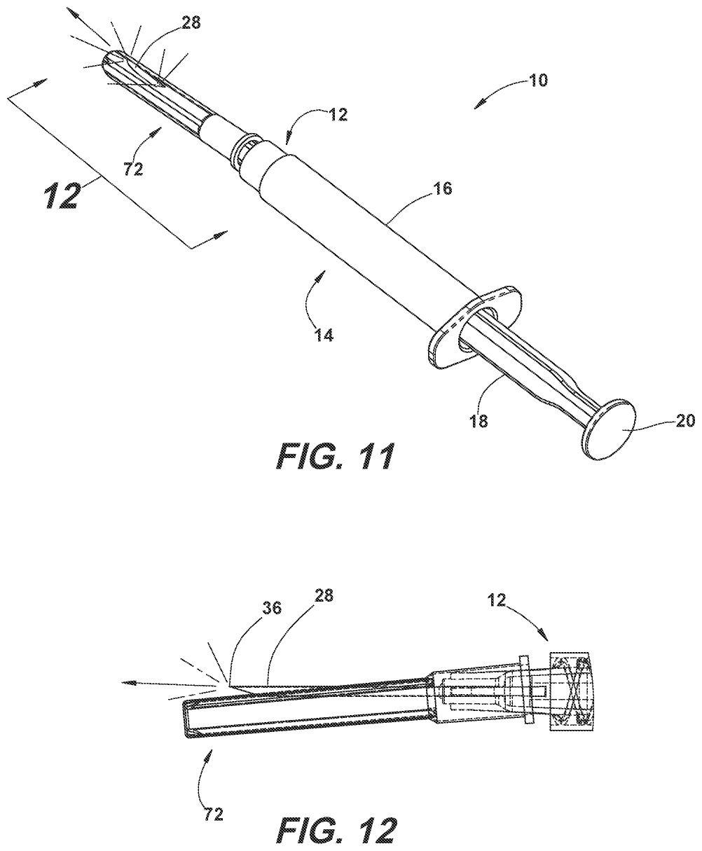

FIG. 11 is a perspective view of a prior art needle and syringe assembly 10 with a mating/demating complementary cap 72 and showing a recently uncovered misalignment needle puncture risk when mating cap 72 onto needle 12 where needle body 28 punctures cap 72 when misaligned during recapping. Plunger stem 18 and end plate 20 are show in a withdrawn position relative to body 16 of syringe 14. This occurrence can yield a needle stick or jab if one wants to recap a needle in order to remove it more easily by the needle going through the cap and into the hand of the user.

FIG. 12 is a side view of the needle and cap puncture risk of FIG. 11 shown by cap 72 and syringe 12. Needle 28 is shown pierced through a sidewall of cover 72 as sharp tip 36 engaged an inner surface of cap 72. Such piercing results from a misalignment of cap 72 during insertion of needle bore 28 of needle assembly 72.

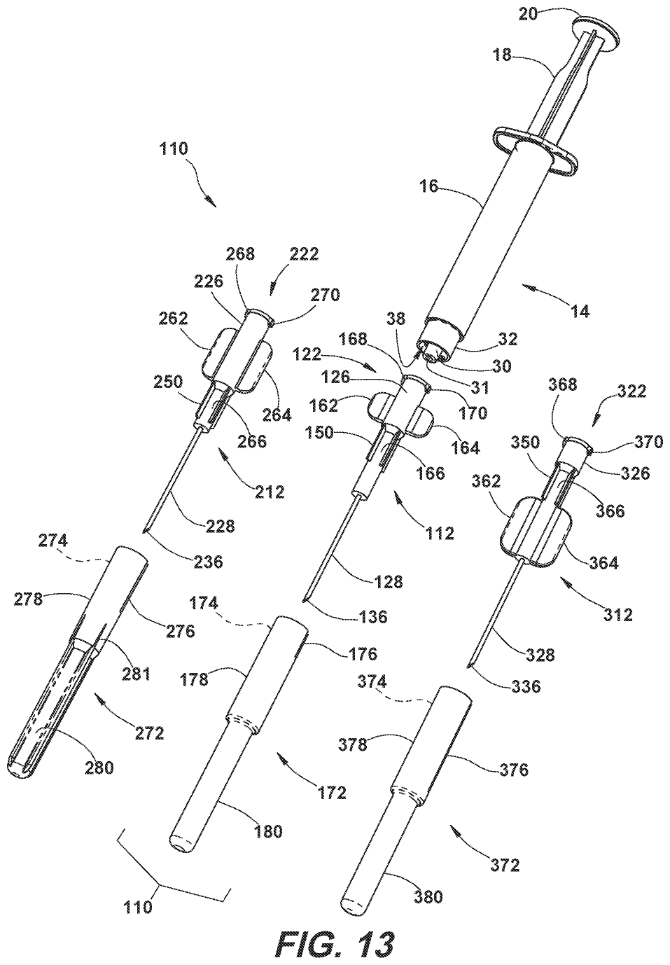

FIG. 13 is a perspective needle end view from above of one overwrap-preventing needle and syringe assembly 110 having a needle 112 and complementary cap 172 removably mated and demated with a complementary syringe 14 and further showing two alternative variation overwrap-preventing needles 212 and 312 and respective complementary caps 272 and 372. Needles 112, 212 and 312 each include wings 162, 164; 262, 264; and 362, 364 configured to facilitate an anatomically manipulation of needle 112, 212, and 312 when mating and demating from syringe 14 that reduces risk of glove overwrap and inadvertent needle puncture, as well as puncture of such needle 128, 228 and 328 through a respective misaligned cap 172, 272 and 372.

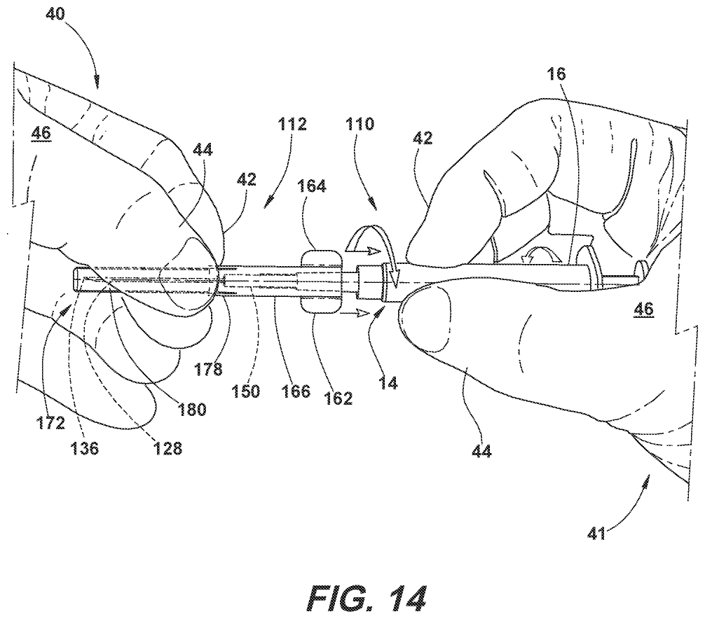

As shown in FIG. 13, needle 112 includes a pair of opposed tabs, or blades 162 and 164 extending radially outwardly from a cylindrical central hub 126 proximate a proximal end and adjacent to luer lock tabs 168 and 170. Luer lock tabs 168 and 170 cooperate with an open cylindrical end of hub 126 to provide a male connector 122. Male connector 122 is configured to mate in complementary relation with a female luer lock connector 38 on syringe 14. Luer lock connector 38 comprises a tapered frustoconical male post 30 provided coaxially within a cylindrical shield, or hub 32. A fluid outlet 31 is provided at a distal end of post 30. Fluid stored in body 16 is delivered from outlet 31 into needle 112 as plunger 18 is urged inwardly within body 16 via a user pressing plunger end 20 into body 16. Wings 162 and 164 are provided on needle base 126 proximate tabs 168 and 170 and longitudinal ribs 150 are provided on reduced-diameter portion 166 on base 126 proximate a distal end of base 126. Placement of wings 162 and 164 proximate connector 122 provides a natural anatomic positioning of syringe 14 and needle 12 as shown in FIG. 14 which keeps gloved fingers from contacting and overwrapping onto ribs 150 while also creating an anatomic positioning of syringe 14 and needle 112 that keeps a user's hand and palm away from sharp tip 136 of hollow needle body 128.

As shown in FIG. 13, needle 112 includes a complementary cap 172 that snaps onto needle 112 to protect users from tip 136 when needle 112 is mated to syringe 14, as well as needle 112 is not mated to syringe 14. Cap 172 comprises a tubular, or cylindrical closed end portion 180 and an enlarged tubular, or cylindrical opposed open end portion 178. A proximal open end of portion 178 includes a pair of diametrically opposed axial slots 174 and 176 formed on an open leading end of portion 178.

Two alternate constructions for needle 112 and cap 172 are shown in FIG. 13 usable with syringe 14. More particularly, needle 212 and complementary cap 272 form one alternate construction, and needle 312 and complementary cap 372 form another alternate construction. Cap 272 includes an array of four equally spaced apart longitudinal splines, or raised longitudinal ridges 281, similar to splines on cap 72 of FIG. 11. Such splines provide a torsional grip when a user affixes a mated cap and needle assembly onto a syringe. In most cases, a user affixes a new needle with the cap attached onto a syringe. Rarely, a user affixes an uncapped needle, as in FIG. 14, onto a syringe. Optionally, caps 172 and 372 can include splines 281, as shown optionally in FIG. 14 on cap 172 below. Further optionally, cap 272 can omit splines 281.

As shown on needle 212 of FIG. 13, a pair of wings 262 and 264 are provided on needle base 226 proximate tabs 268 and 270 and longitudinal ribs 250 are provided on reduced-diameter portion 266 on base 226 proximate a distal end of base 226. Placement of wings 262 and 264 proximate connector 222 provides a natural anatomic positioning of syringe 14 and needle 12 similar to that shown in FIG. 14 which keeps gloved fingers from contacting and overwrapping onto ribs 250 while also creating an anatomic positioning of syringe 14 and needle 112 that keeps a user's hand and palm away from sharp tip 136 of hollow needle body 128 during removal, as well as attachment. However, wings 262, 264 and hub 226 are provided with a longer axial length than wings 162, 164 and hub 126 in order to provide increased tactile surface area for a user's fingers when grasping and rotating needle 212 in a manner similar to that depicted in Figure '14.

Cap 272 for needle 212 has slots 274 and 276 that complement tabs 262 and 264 and are elongated over slots 174 and 176 for needle 112, as shown in FIG. 13. Slots 274 and 276 are provided in a proximal end of enlarged tubular cap portion 278 such that needle tube 228 and tip 236 are received in assembly within reduced diameter, or stepped-down tubular portion 280. Such slots 274 and 276 cooperate with elongated tabs 274 and 276 to help better align needle tube 228 when being inserted into cap 272 which reduces risk of inadvertent puncture of cap 272 similar to what is shown with cap 72 in FIGS. 11 and 12.

As shown on needle 312 of FIG. 13, a pair of wings 362 and 364 are provided on needle base 326 proximate tabs 368 and 370 and longitudinal ribs 250 are provided on reduced-diameter portion 366 on base 326 proximate a distal end of base 326. Placement of wings 362 and 364 distally of connector 322 provides an optional natural anatomic positioning of syringe 14 and needle 12 similar to that shown in FIG. 14 which keeps gloved fingers from contacting and overwrapping onto ribs 350 because such finger, or digit engages with wings 362 and 364 while also creating an anatomic positioning of syringe 14 and needle 112 that keeps a user's hand and palm away from sharp tip 136 of hollow needle body 128

Cap 372 for needle 312 has slots 374 and 376 that complement tabs 362 and 364 and are also elongated over slots 174 and 176 for needle 112, as shown in FIG. 13. Slots 374 and 376 are provided in a proximal end of enlarged tubular cap portion 378 such that needle tube 328 and tip 336 are received in assembly within reduced diameter, or stepped-down tubular portion 380. Such slots 374 and 376 cooperate with elongated tabs 374 and 376 to help better align needle tube 328 when being inserted into cap 372 which reduces risk of inadvertent puncture of cap 372 similar to what is shown with cap 72 in FIGS. 11 and 12. Location of tabs 362 and 364 distally of ribs 350 on hub 366 enhances alignment features with slots 374 and 376 as needle tube 328 and tip 336 are inserted within cap 372.

FIG. 14 is a perspective view of a user mating the one overwrap-preventing needle 112 of FIG. 12 onto a syringe 14 while encased in a cap 172 of needle/syringe assembly 110. More particularly, specific orientation of tabs, or wings 162 and 164 favors a user grasping needle 112 encased in cap 172 with a left hand 40 and syringe 14 with a right hand 41 (or vice versa) while wearing gloves 46 particularly with thumb, or digit 44 of left hand 40 engaging wing 162 and fore finger, or digit 42 engaging wing 164. This biomechanical mating configuration using both hands 40 and 41 when needle cap 172 is affixed onto needle 112 protects hand 40 when mating onto syringe 14. Likewise, thumb 44 and fore finger 42 of right hand 41 grasps body, or barrel 16 of syringe 14. Such human factors optimization becomes natural and preferred when a user grasps needle 112 and cap 172 along with syringe 14 to mate a capped needle 112 onto syringe 14 as tabs 162 and 164 are provided in such orientation to provide rotational leverage when mating needle 112 onto syringe 14, overcoming a natural tendency for a user to grasp any ribs on a needle hub, such as ribs 34 of FIG. 2. Such mating technique is typically used when installing an new, unused needle 112 and cap 172 as they are already hermetically affixed together in a sterile package (not shown), thereby encasing and protecting a sharp needle tip 236 of a needle tube 128 within a distal closed end reduced diameter tubular portion while hub 150 is encased with an enlarge tubular portion 178. Reduced diameter portion 166 of needle 112 is also encased within portion 178 while wings 162 and 164 are exposed outside of cap 172.

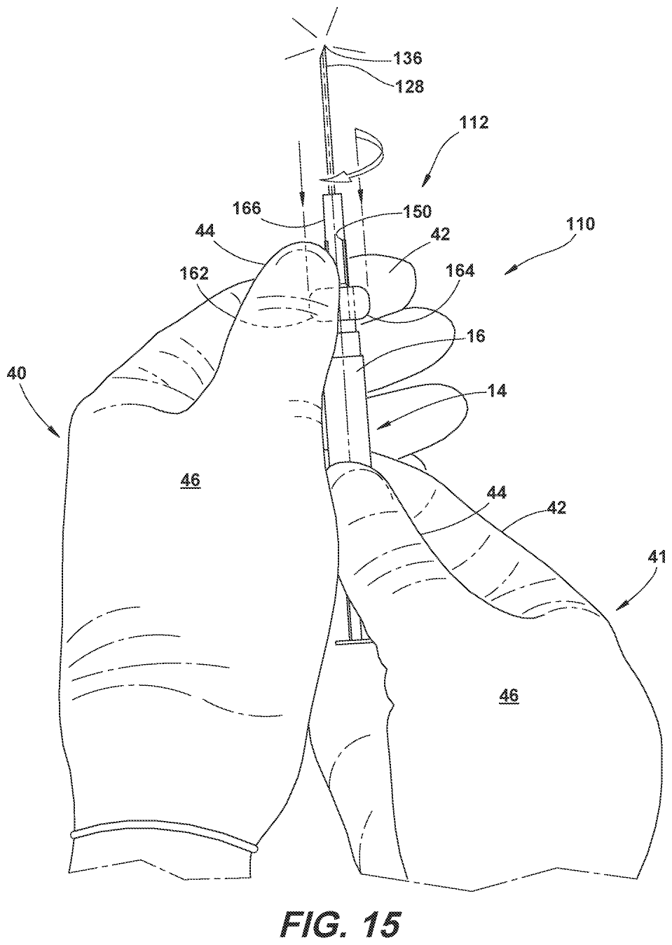

FIG. 15 is a perspective view of a user mating, or affixing the one overwrap-preventing needle 112 of FIG. 13 onto a syringe 14 of needle/syringe assembly 110. More particularly, specific orientation of tabs, or wings 162 and 164 favors a user grasping needle 112 with a left hand 40 and syringe 14 with a right hand 41 (or vice versa) particularly with thumb 44 of left hand 40 engaging wing 162 and fore finger 42 engaging wing 164. It has been discovered that such design, with an exposed sharp needle tip 136, provides an ergonomic grasping configuration that naturally motivates a user to orient needle tip 136 away from a user and a user's hands 40 and 41 encased in protective gloves 46. Likewise, thumb 44 and fore finger 42 of a right hand 41 grasps body 16 of syringe 14. Such human factors optimization becomes natural and preferred when a user grasps needle 112 and syringe 14 when tabs 162 and 164 are provided in such orientation, overcoming a natural tendency for a user to grasp any ribs on a needle hub, such as ribs 34 of FIG. 2. Such natural ergonomic orientation serves a purpose to make it tactilely natural for a user to “point away” a sharp needle tip 136 from needle tube 128. As a consequence, a user is protected from inadvertent jabs from needle tip, or end 136 because tip 136 is never pointed at a human extremity, but is pointed away from hands 40 and 41 and the body, and a user's gloves 46 are never forcibly engaged with any ribs 150 on a hub 166 of needle 112, thereby preventing the wrap-around entanglement shown with the prior art needle discovered and shown in FIGS. 2-8, as well as preventing the risky anatomic positioning detailed in FIGS. 9 and 10. Such natural ergonomic orientation reduces the risk of puncture by having the needle pointed away from the user by changing the biomechanics of how the needle 112 is more naturally and easily manipulated and mounted, or mated onto a syringe 14. This configuration of the flanges results in behavior modification of the user providing a safer work environment.

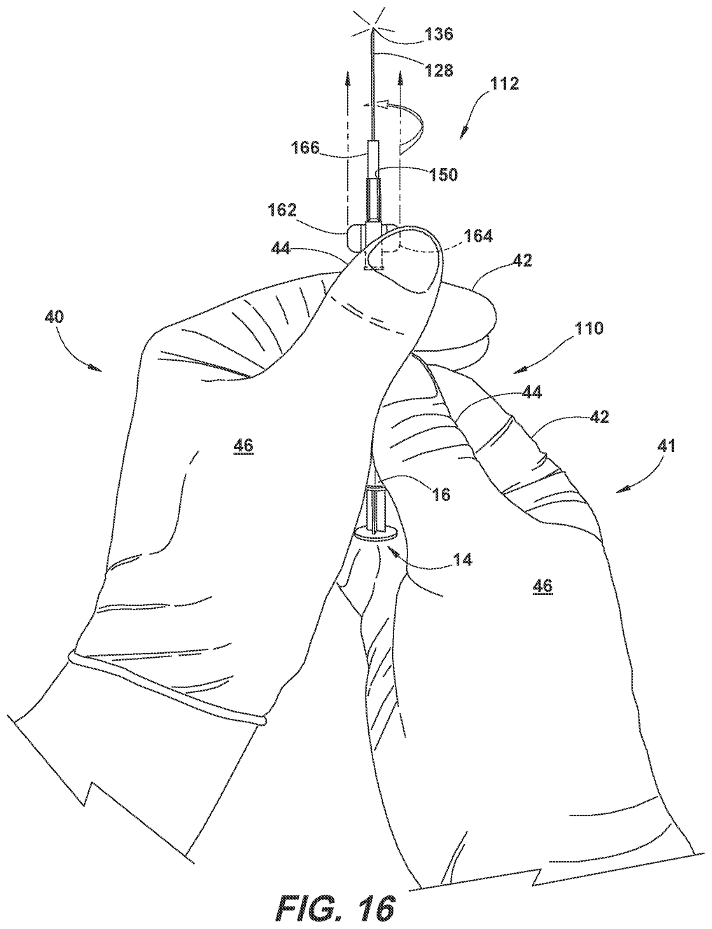

Furthermore, such natural ergonomic orientation of FIG. 16 serves a purpose to make it tactilely natural for a user to “point away” a sharp needle tip 136 from needle tube 128. As a consequence, a user is protected from inadvertent jabs from needle tip, or end 136 because tip 136 is never pointed at a human extremity, but is pointed away from hands 40 and 41 and the body, and a user's gloves 46 are never engaged using forcible loading with any ribs 150 on a hub 166 of needle 112, thereby preventing the wrap-around entanglement shown with the prior art needle discovered and shown in FIGS. 2-8, as wells as preventing the risky anatomic positioning detailed in FIGS. 9 and 10. Wings 42 and 44 simply prevent any opportunity for overwrap on ribs 166 as they provide a positive tactile engagement surface for finger 42 and thumb 44. Such natural ergonomic orientation also reduces the risk of puncture by naturally pointing the needle away from the user by changing the biomechanics of how the needle is easily manipulated and removed when cap 172 is removed, as shown below in FIG. 15. Optionally, it is understood that the anatomic orientation of an operator shown in FIG. 15 can also be used to remove an uncapped needle using both hands and mitigating risk of needle puncture by naturally adopting the biomechanically preferential mating between a user's left and right hands that points away the needle tip from a user and the user's hands.

FIG. 16 is a perspective view of a user demating, or removing the one overwrap-preventing needle 112 of FIG. 13 after already being installed onto a syringe 14 of needle/syringe assembly 110. More particularly, specific orientation of tabs, or wings 162 and 164 favors a user grasping needle 112 with a left hand 40 and syringe 14 with a right hand 41 (or vice versa) particularly with thumb 44 of left hand 40 engaging wing 162 and fore finger 42 engaging wing 164. It has been discovered that such design, with an exposed sharp needle tip 136, provides an ergonomic grasping configuration that naturally motivates a user to orient needle tip 136 away from a user and a user's hands 40 and 41 encased in protective gloves 46. Likewise, thumb 44 and fore finger 42 of a right hand 41 grasps body 16 of syringe 14. Such human factors optimization becomes natural and preferred when a user grasps needle 112 and syringe 14 when tabs 162 and 164 are provided in such orientation, overcoming a natural tendency for a user to grasp any ribs on a needle hub, such as ribs 34 of FIG. 2. Such natural ergonomic orientation serves a purpose to make it tactilely natural for a user to “point away” a sharp needle tip 136 from needle tube 128. As a consequence, a user is protected from inadvertent jabs from needle tip, or end 136 because tip 136 is never pointed at a human extremity, but is pointed away from hands 40 and 41 and the body, and a user's gloves 46 are never forcibly engaged with any ribs 166 on a hub 150 of needle 112, thereby preventing the wrap-around entanglement shown with the prior art needle discovered and shown in FIGS. 2-8, as well as preventing the risky anatomic positioning detailed in FIGS. 9 and 10. Such natural ergonomic orientation reduces the risk of puncture by having the needle pointed away from the user by changing the biomechanics of how the needle is more naturally and easily manipulated and removed. This configuration of the flanges results in behavior modification of the user providing a safer work environment.

Furthermore, such natural ergonomic orientation of FIG. 16 serves a purpose to make it tactilely natural for a user to “point away” a sharp needle tip 136 from needle tube 128. As a consequence, a user is protected from inadvertent jabs from needle tip, or end 136 because tip 136 is never pointed at a human extremity, but is pointed away from hands 40 and 41 and the body, and a user's gloves 46 are never engaged using forcible loading with any ribs 166 on a hub 150 of needle 112, thereby preventing the wrap-around entanglement shown with the prior art needle discovered and shown in FIGS. 2-8, as wells as preventing the risky anatomic positioning detailed in FIGS. 9 and 10. Wings 42 and 44 simply prevent any opportunity for overwrap on ribs 166 as they provide a positive tactile engagement surface for finger 42 and thumb 44. Such natural ergonomic orientation also reduces the risk of puncture by naturally pointing the needle away from the user by changing the biomechanics of how the needle is easily manipulated and removed when cap 172 is removed, as shown below in FIG. 15. Optionally, it is understood that the anatomic orientation of an operator shown in FIG. 15 can also be used to remove an uncapped needle using both hands and mitigating risk of needle puncture by naturally adopting the biomechanically preferential mating between a user's left and right hands that points away the needle tip from a user and the user's hands.

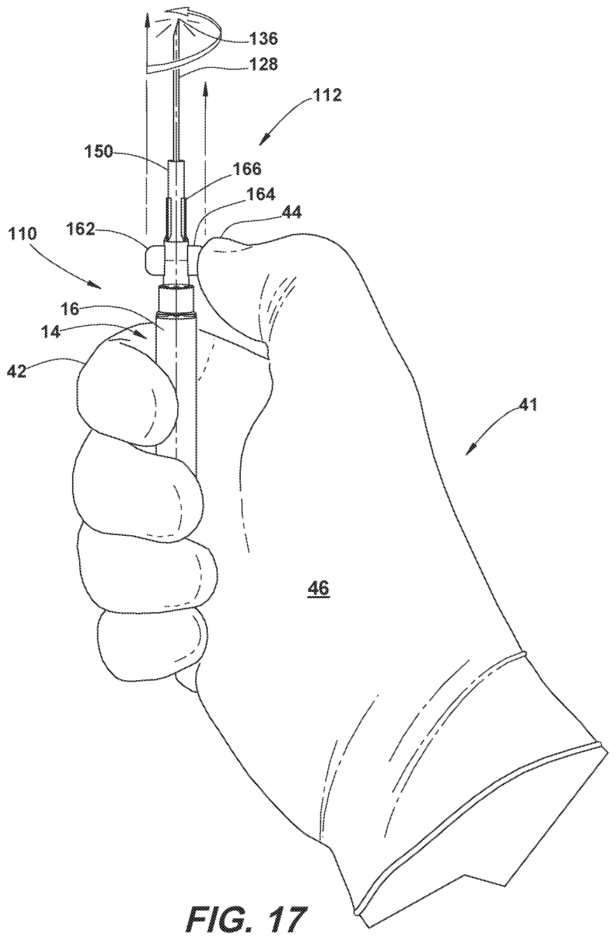

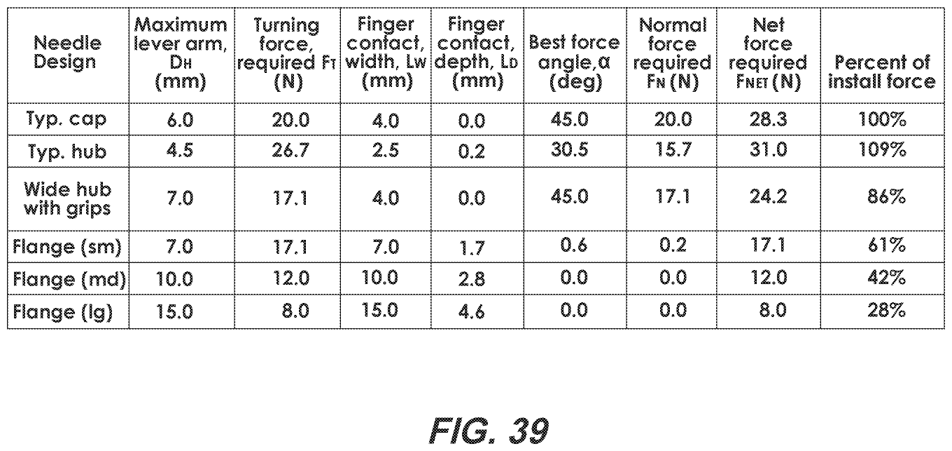

FIG. 17 a perspective view of a user demating the one overwrap-preventing needle 112 of FIG. 13 from a syringe 14 in a single-handed manner that reduces the risk of puncture by having sharp needle tip 136 on needle tube 128 pointed away from the user. By providing a natural and biomechanically preferential mating between a user's single hand 41 with needle and syringe assembly 110 (without a cap), a user has no need to present a user's other hand (in this case, hand 40 of FIG. 16) anywhere near sharp needle tip 136, thereby mitigating any risk of inadvertent contact between tip 136 and a user as tip 136 is always pointed away from a user. Hence, hand 41, encased in protective glove 46, grasps barrel 16 of syringe 14 (on assembly 110) with folded fingers, such as index finger 42. Thumb 44 is naturally presented in such position to tactilely engage with wing 164 opposite wing 162 to rotate and remove needle 112 from syringe 14. Additionally, such natural biomechanical presentment of needle and hub assembly 110 with sharp tip 136 pointed away from a user places thumb 44 in an orientation that simply cannot engage with force and overwrap with longitudinal ribs 166 on hub 150. Ribs 166 cooperate to ensure secure and guided over-engagement of a cap onto needle 112. An over-engagement is difficult because the threads override (pop through) or stop because of an end stop. Flanges provided greater counter-torque than loaded engagement limited by the override when loading (SEE FIG. 39/TABLE). This ensures greater removal torque than loading torque.

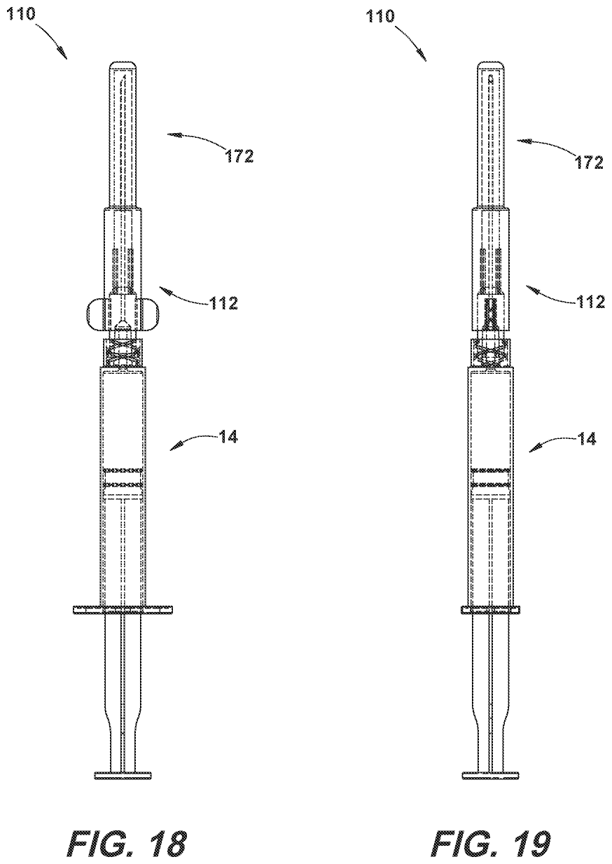

FIG. 18 is a front elevational view of the one overwrap-preventing needle and syringe assembly 110 of FIGS. 13-17. More particularly, cap 172 is shown seated, or locked axially onto and over needle 112. Needle 12 is shown attached to syringe 14. FIG. 19 is a similar corresponding right side view of the one overwrap-preventing needle 112 and syringe 114 of assembly 110 shown in FIGS. 13-17 with cap 172 affixed atop and over needle 112.

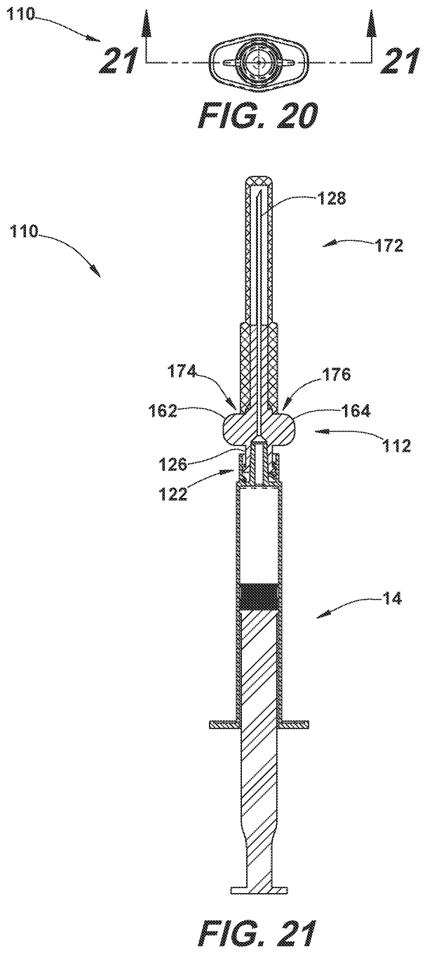

FIG. 20 is a top end view of the one overwrap-preventing needle and syringe assembly 110 of FIGS. 13-18.

FIG. 21 is a vertical sectional view of the one overwrap-preventing needle and syringe assembly 110 of FIG. 13 in assembly taken along line 20-20 of FIG. 20. Needle 112 is affixed, or locked onto syringe 14 using a luer lock connection comprising a female sealing connector 122 on syringe 14. Needle base 126 of needle 112 is affixed onto connector 122 with wings, or tabs 162 and 164 provided proximate syringe 14 and tabs 162 and 164 received into slots 174 and 176, respectively as cap 172 is affixed or snap-fit over needle bore 128.

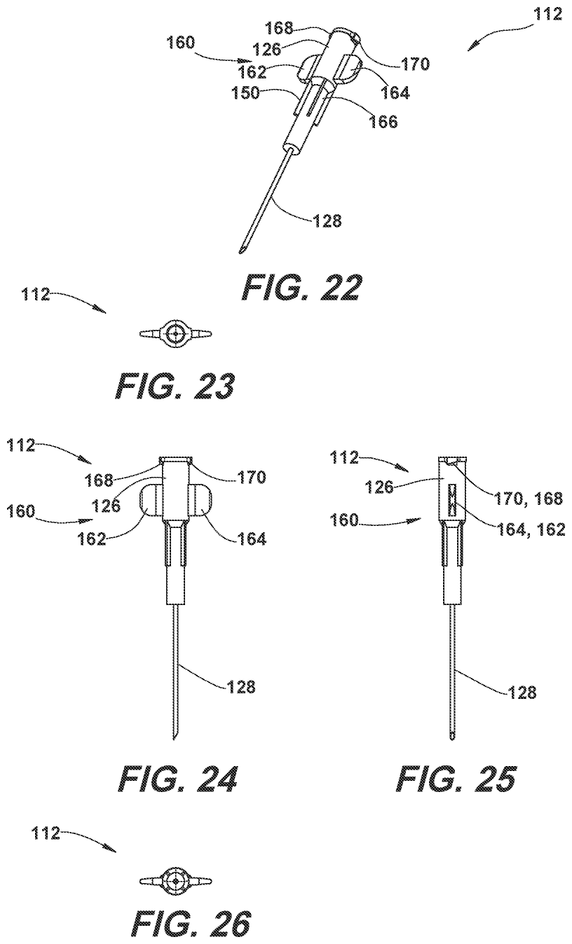

FIG. 22 is a needle end perspective view from above of the one overwrap-preventing needle 112 of FIGS. 13-21. More particularly, male luer connector members 168 and 170 are provided on a proximal end of hub 126 with tabs 162 and 64 provided distally on hub 126 spaced from male connectors 168 and 170. Needle tactile user interface 160 comprises wings 162 and 164 on hub 126 which are provided proximate connectors 168 and 170, while ribs 150 are provided along a distal portion of hub 126, proximate needle bore 128.

FIG. 23 is a connector end view of the one overwrap-preventing needle 112 of FIG. 22.

FIG. 24 is front elevational view with the needle pointed down of the one overwrap-preventing needle 112 of FIGS. 13-22. More particularly, male luer connectors 168 and 170 on hub 126 are shown on a proximal portion of hub 126 with wings 162 and 164 shown on a distal portion of hub 126 next to needle bore 128 in order to provide user interface 160.

FIG. 25 is a right side view of the needle 112 of FIG. 24 further illustrating details of connectors 168 and 170, hub 126, interface 160 with wings 162 and 164 and needle bore 128.

FIG. 26 is a needle end view of the one overwrap-preventing needle 112 of FIGS. 21-25.

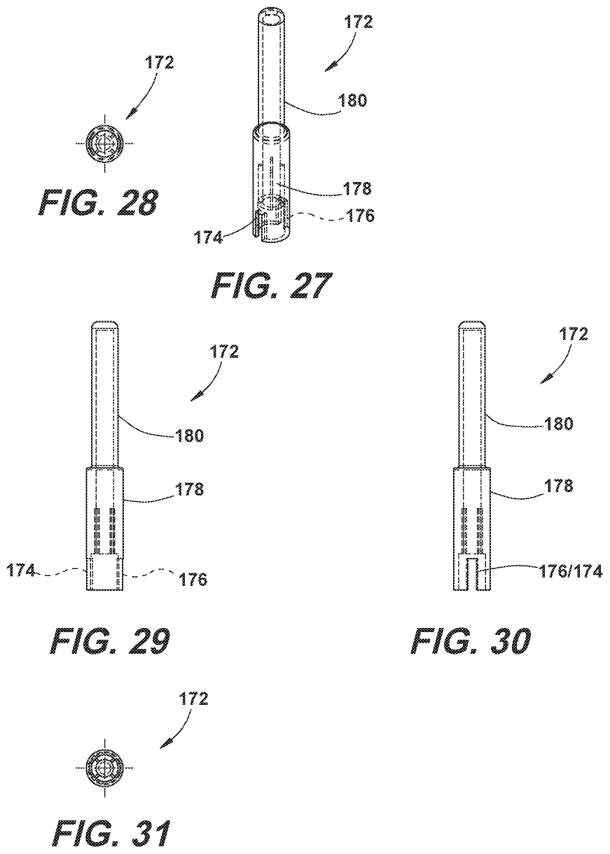

FIG. 27 is a perspective view from a closed end of a cap 172 for the overwrap-preventing needle 112 of FIGS. 13-26. More particularly, enlarged tubular portion 178 includes axial slots 174 and 176 and has an open leading proximal end with reduced diameter tubular portion 180 provided distally of portion 178.

FIG. 28 is a top end view of the end cap 172 of FIG. 27.

FIG. 29 is a front elevational view of the end cap 172 of FIG. 28. Portions 178 and 180 are further shown.

FIG. 30 is a right side elevational view of the end cap 172 of FIG. 29 showing slots 174 and 176 in portion 178 spaced from portion 180.

FIG. 31 is a bottom end view of the end cap 172 of FIG. 29.

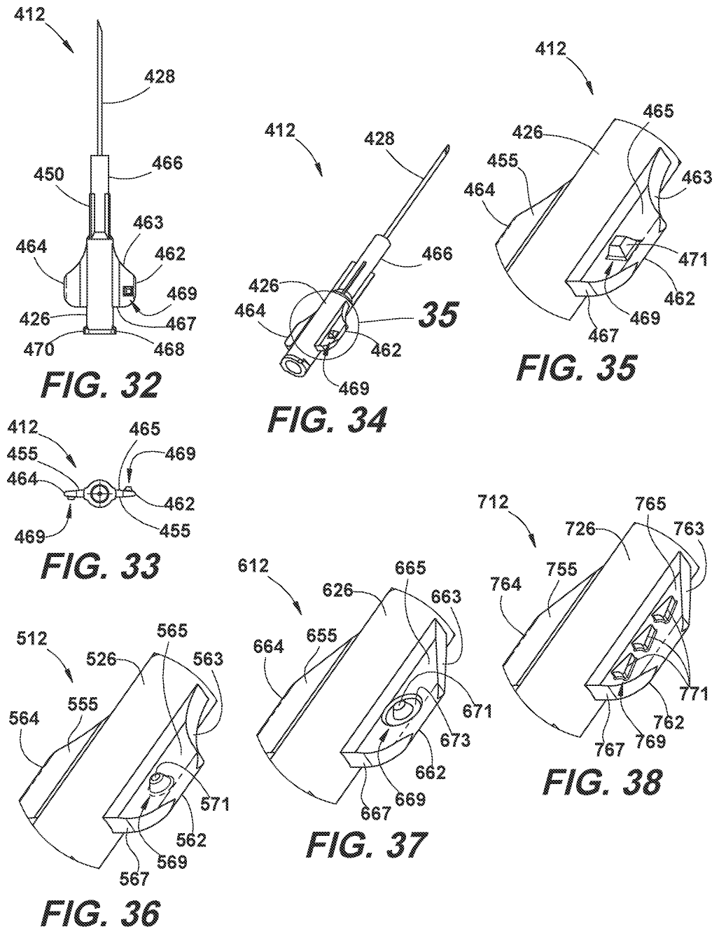

FIG. 32 is a front elevational view of an alternative variation overwrap-preventing needle 412 similar to that depicted in FIGS. 13-23, but having a curved leading edge 463 with a delta configuration and a tactilely detectible surface feature, or tactile element 469 on a removal leverage surface 465 (see FIG. 35) of each tab, or rotational lever 462 and 464. Hollow needle body 428 is carried by reduced diameter portion 466 of base 426. Longitudinal ribs 450 on portion 466 serve to mate with a cap as a cap mounting surface. Luer lock members 468 and 470 are provided on base 426. Finally, tabs, or paddles 462 and 464 each have an angled distal edge 463 and a transverse or perpendicular proximal edge 467. It has been observed that such a delta configuration provided by angled distal edge 46 deters a user from approaching tabs 462 and 464 from a distal direction. Instead, anatomic and visual cues encourage a user to approach tabs 462 and 464 from a proximal direction as shown in FIGS. 15 and 16.

FIG. 33 is a connector end view of the one overwrap-preventing needle 412 of FIG. 32 showing the diametrically opposed radially outwardly extending orientation of tabs 462 and 464. Tactilely detectible surface feature, or tactile element 469 is provided on each tab 462 and 464 in a location that identifies a removal surface 465 of each tab 462 and 464. No such feature is provided on the opposing mating surface 455. Optionally, a single tactile element, or raised button 469 can be provided solely on a single tab 462.

FIG. 34 is a connector end perspective view from above of the one overwrap-preventing needle 412 of FIGS. 32-33. More particularly, needle body 428 is shown mounted to portion 466 and base 426 in perspective view. Tactile element, or raised button 469 is shown on tab 462 to identify a removal interface, whereas tab 464 has no such feature because the raised button is on an opposed side. Hence, raised buttons 469 are provided solely on a unwind, or removal surface of each tab 462 and 464 in a region of base 426.

FIG. 35 is an enlarged view of the curved leading edge delta configuration for the tabs 462 and 464 on needle 412 taken from the encircled region 35 of FIG. 34. Tactile element 469 in one form is a rectangular, trapezoidal raised button 471 provided on a drive removal surface 465 on tab 462. Edges 463 and 467 extend outwardly from base 426. Mating, or mounting drive surface 455 provides no such feature and gives no tactile feedback or visual indication to a user engaging such surface 455 when mounting and engaging a needle 412 onto a syringe. Drive surfaces 455 and 465 each provide a finger, or digit pad for a user's finger when mating and de-mating a needle from a syringe. Such surfaces 455 and 465 can be smooth, save for the tactile element 465 on drive surface 465. Optionally, surface 465 can be a frictionable surface that further enhances finger engagement and grip when removing a needle. Further optionally, any of the tactile elements shown in FIGS. 36-38 can be provided along with or separate from a frictionable surface. Even further optionally, scribed lines, grids, raised diamond patterns, undulations or any other combination of visually identifiable or tactilely discernible elements can be provided on the drive removal surface 465 on tab 462. Further optionally, the tactile element can be provided on the drive mounting surface 455 of tab 464 and no such surface is provided on the drive removal surface 465 of tab 4651 wherein a user is trained to identify the visually discernible and/or tactilely discernible elements on the drive mounting surface.

Positioning of tactile feedback device, or tactile output 469 on tabs 462 and 464 of FIG. 35 on removal drive surfaces 465 provide both a visual and tactile input to a user that is taught to engage such button 471 when removing a needle 412 from a syringe. In most instances, a user will mate needle 412 onto a syringe while still protected by a cap, as shown in FIG. 14. However, the mating configuration of FIG. 15 can also be used in some instances. Training in combination with visual and tactile feedback will lend itself to users always pointing an exposed needle away from a user's hands 40 and 41, especially in combination with the delta edge 462 shown in FIG. 32. Left-handedness does not change the incentive provided to a user to place a finger, or thumb onto tactile output 469 as both a visual and tactile feedback to the user.

FIG. 36 is an enlarged view corresponding with that shown in FIG. 35, but showing a first alternative curved leading edge delta 563 configuration for one tab 565 having a round raised button 571 providing tactile element 569.

FIG. 37 is an enlarged view corresponding with that shown in FIG. 35, but showing a second alternative straight leading edge delta 663 configuration for one tab 665 having a round raised button 671 within a cylindrical button-shaped depression. 673

FIG. 38 is an enlarged view corresponding with that shown in FIG. 35, but showing a third alternative straight leading edge delta 763 configuration for one tab 765 having a linear array of three raised delta darts 771.

FIG. 39 is a Table illustrating biomechanical interface features of various needles. More particularly, biomechanical estimates of force are tabulated for a range of effective lever arms for a typical cap, typical hub, wide hub, and various sized/length flanged, or tabbed needle hubs.

The present problem and embodiments shown in FIGS. 1-39 that solves such problem does the following in order to increase safety. First, it allows for more torque to be applied to remove the needle safely, as described below in greater detail. Secondly, the flanges are small enough that they will not obstruct the surgeon or users' visual axis to the area since they are low profile and the needle can be spun if they are obstructing a part of the view even if the user is injecting into a small orifice or space.

With regards to ergonomics, the flanges almost automatically change how the user will remove the needle based on natural human ergonomics. Currently, users will naturally carry out an overhand grasp with the needle pointed at the palm of the user and it is hidden under the knuckles and hand. This technique has risk in two ways. First, it leaves the sharp end of a needle pointed at the user. Secondly, the needle is hidden where the user cannot see it. Additionally, if the user wants to reapply the cap over the needle to make its removal easier, as a capped needle reduces the difficulty of unscrewing the needle, the reapplication of the cap increases the risk. This is likely why the cap is square in shape, not round, which allows for easier removal/unscrewing of the needle off of the syringe. The action of recapping a needle is a known risk and a “one handed technique” is recommended, but not always used, and is not always practical. Secondly, even if the cap is placed onto the needle, it has to be push on with additional force in order to get it to “click” into place over the hub so it can be unscrewed. This likely why there are triangular shaped elevations on the hub which allow for orientation of the needle in the cap and for it to “lock into place” so the needle can't just fall out of the cap when it's opened from the packaging. The capping of the needle is usually done inside the palm as the needle and cap are held in the way described above but a 18 gauge needle is stiff enough and strong enough to pierce through the needle cap and then “stick” the user (this has happened to the present inventor).

Further with regards to ergonomics, the flanges change the ergonomics by almost instinctively encouraging the user to point the needle away from the user with the left hand (in a right handed person) grasping the flanges with an up facing open palm technique (in a supination position) while the dominant right hand holds the syringe as it normally would in a downward palm facing (pronation) position. One could call this or name this a “in parallel technique” compared to a more ‘appositional traditional technique’ where the two hands are facing each other with the needle between them. This technique then allows the left hand to hold the flanges while the right hand unscrews the syringe using a supination motion. This supination motion with the right hand will allow for a greater amount of force to be applied to a needle, which if flanges are attached, will allow for successful removal because the needle is screwed onto the hub with a pronation motion with either hand. Supination is a more powerful motion with the human hand than pronation. The ability to use a more ergonomic motion that generates more force is one safety mechanism. It is worth noting that Pronation is takes about 80% the strength of Supination.

Yet another ergonomic consideration, this more intuitive and natural set up of the proposed present design also allows for the needle never to be “hidden” inside the palm which increases safety by first exposing the needle along its entire length so it is visible, secondly, the tip of the needle is always visible, and thirdly the tip of the needle is pointed away from the user.

Yet another ergonomic point, one previously known effort has multiple ribs on the hub of a needle that would allow for the user to be able to apply more force in unscrewing the needle compared to screwing the needle on (U.S. Pat. No. 9,238,111). However, this effort still carries the issue of the ergonomics where the user will intuitively place the needle hidden in their palm with an overhand technique to unscrew it. Hence, this increases their risks because they will be unable to see the needle inside their hand much like the prior art current needle set up and design (of FIG. 1) currently on the market. Furthermore, the present design is described as if the needle is screwed on bare without a cap in order for its mechanical mating to work which is not necessary. Although is can still happen, it is preferred to be screwed on with a cap; therefore, the small ribs may not provide enough friction to unscrew it. It also cannot be un-screwed pointing away from you as enough force cannot generally be applied.

In the event that a user wants to reapply the needle without the cap on, such as in a situation in the operating room, this needle design would allow for the needle to be screwed onto the syringe with the needle tip still being pointed away from the user without the cap being on the needle, as shown in FIG. 15. No other current needle design would allow for a user to apply the needle and ensure an airtight and watertight seal that this design enables. All other designs make it very prohibitive for reapplying the needle to the syringe with the needle tip pointed away from the user because the user cannot generate enough force between their fingertips and thumb while squeezing the small needle hub (of hub 26 in FIG. 1 while the syringe is tightened and screwed onto the needle. The needle simply rotates in the users gloved hand which increases risks. The user can screw an uncapped needle on with it pointed at the user's palm, but this is a very high risk situation because an uncapped needle is in the user's palm.

The new cap designs shown herein also allow for recapping of the needle should the needle need to be recapped at any time. It allows the needle flanges to be exposed, but the cap still covers the needle for safety as one would do in the operating room setting so that no needle tip is exposed to injure a health care worker. Lastly, the slots in the cap that allow it to fit around the flanges also provide additional safety if recapping is required. It is understood that the goal is to minimize or eliminate those situations. Individuals can use those slots to help guide the needle tip into the proper location.

As the US workforce ages, including physicians, nurses, and other healthcare providers, the incidence of arthritis and neuropathy of the extremities including in hands resulting from diabetes, as well as other medical conditions causing such increases in incidence. This will impact our workforce. These individuals need to be able to manipulate their tools such as a needle with efficiency and safety. A neuropathy (decreased sensation or touch) that can occur with conditions such as diabetes or carpal tunnel syndrome will cause decreased tactile sensation which will make determining what part of the needle they are touching difficult and they may not be able to feel the finer edges or small ridges of any of the prior art needles increasing the risk to them or their patients in a health care worker's case. The same applies for patients receiving home healthcare, or self-administered care. This device will allow individuals with decreased tactile sensation from neuropathies to be able to feel the edges with greater certainty. Furthermore, patients with arthritis, both osteoarthritis and rheumatoid arthritis, could have degenerative joint disease of their hands making manipulation of such small devices as needles more difficult. Patients or health care workers with rheumatoid or osteoid arthritis can have fingers deviate severely laterally (toward the 5th digit/pinky finger) (ulnar deviation) and will not be able to manipulate the small hub of the needles previously described/of prior art and remove the needle safely. The present designs reduce these risks and makes it more manageable without creating an unusable product or resulting in cumbersome manufacturing.

Furthermore, in regard to the above previous point, medications that are injectable are being given at an increasing rate in the at home setting by patients. Medications include both anticoagulants (blood thinners prior to surgery) and diabetic medication such as insulin are injected underneath the skin. There are other medications in the rheumatologic field that are also given by injection by the patient at home. These individuals may suffer from one or multiple of the above conditions concurrently. This will make applying these medications more challenging for these individuals unless they have a needle that can be easily manipulated should they have to be required to draw the medication up themselves. The present designs allow for safer use in patients who need home medications for diabetes (insulin) injections or rheumatoid or autoimmune disease who do injections at home.

Some medications such as Botox need to be drawn up in an 18 gauge needle because there is a theory that they should not be drawn up in a smaller gauge needle as it may shear some of the proteins of the toxin, thereby rendering the medication less effective. Therefore, removal of the medication from the bottle and the large 18 gauge needle and subsequent removal of the needle is required so that we can reapply the smaller needle for injection into the patient. Sometimes the needle is left in the bottle to reduce the number of times the needle is punctured into the bottle. (Don't know if we should leave this last sentence (this is a practice that is done but discouraged by the government agencies)

It is also understood that companies should acknowledge that the mere unscrewing of a needle is difficult, otherwise the cap would not be square and have a locking mechanism to the needle hub in order to allow for more mechanical advantages for unscrewing.

There exist other side benefits of increased safety from the proposed patent. For example, if a provider is allergic to a medicine in which they are drawing up into a syringe and then sticks themselves with the needle they now have exposed themselves to a medicine they are allergic to.

An inadvertent stick (into the body) of a health care worker trying to recap a needle contaminates the drug with human contaminate and the drug must be discarded which could be very expensive with some drugs (i.e. Botox™ is $600 per bottle).

The needle design in U.S. Pat. Nos. 9,238,111 and 2,564,804 have a flaw in that in the current health care field we wear gloves that are usually nitrile which do not fit snuggly to the skin to the health care provider. Therefore, based on such designs unscrewing the needle would cause the gloves to get “wrapped around” the needle without unscrewing the needle from the syringe making the situation more dangerous because now you have your finger trapped in a glove wrapped around a needle with potentially medicine or even biohazards waste on it. The present designs would significantly reduce or eliminate this issue. With the prior art descriptions, this would be less of an issue with the use of latex gloves, which were used much more in the past, but nitrile gloves are nearly ubiquitous in patient rooms and offices (except in the operating room) because of the issue of latex allergy in many patients.

Another issue results when trying to unscrew a needle as shown in U.S. Pat. Nos. 9,238,111 and 2,564,804. More particularly, one would have to likely “overhand” the needle in order to unscrew it which causes the needle to be pointed toward the palm of the health care provider trying to unscrew it. This is issue is previously described above.

Original size proposals for the present designs had flanges of a determined length, thickness, and stiffness. More particularly, the tabs were sized at 0.25″×0.5″ with the thickness at the base as 0.075″, and they were engineered to generate enough anticipated torque to realize 0.07N-m (0.6 in-lbs), which would allow nearly all finger sizes to have a sufficient platform to unscrew the needle regardless of physical limitations/disabilities as described above. However, the table in FIG. 30 lists more data. The table shows the flanges allow for more force for removal of the needle than to load (install) it as limited by a stop or thread override.

If the removal of the needle from a syringe is made more safe, then the ability to remove the needle can enhance a reduction in medical waste. The present designs could also allow for reduction in medical waste produced if it allows for more safety removal of needles so that a syringe and needle do not have to be put into a sharps medical waste box. The needle could be put in alone and the syringe into regular waste.

In compliance with the statute, the subject matter disclosed herein has been described in language more or less specific as to structural and methodical features. It is to be understood, however, that the claims are not limited to the specific features shown and described, since the means herein disclosed comprise example embodiments. The claims are thus to be afforded full scope as literally worded, and to be appropriately interpreted in accordance with the doctrine of equivalents.

I/We claim:

1. A needle assembly, comprising:
a needle cannula having a proximal end and a distal end with a puncture tip;
a hub having a proximal end and a distal end, the hub supporting the needle cannula at a distal end, a twist fitting connector provided at the proximal end; and
a pair of opposed radially outwardly extending lever arms each having a digit pad configured on the hub in an anatomically preferential position when the needle is affixed to a syringe with the connector such that a user is induced to palm-grasp a barrel of the syringe in a manner that points the puncture tip away from a user when receiving at least one digit on at least one of the digit pads during mating and demating from the syringe.

2. The needle assembly of claim 1, wherein at least one of the lever arms has a visual and/or tactile feedback indicia on one of a removal surface of the digit pad and a mounting surface of the digit pad.

3. The needle assembly of claim 2, wherein the threaded connector is a clockwise connector and the removal surface has indicia corresponding with a user engaging the surface and the indicia in a counterclockwise direction.

4. The needle assembly of claim 1, wherein the indicia comprises a raised projection.

5. The needle assembly of claim 4, wherein the raised projection comprises at least one raised button.

6. The needle assembly of claim 5, wherein the raised projection is provided by an array of raised projection.

7. The needle assembly of claim 4, wherein the indicia comprises a recess.

8. The needle assembly of claim 1, further comprising a cap having a closed distal end portion configured to receive and protect the puncture tip of the needle, an open leading end portion of the cap, and a pair of diametrically opposed axial slots contiguous with the open leading end portion of the cap each configured to received one of the lever arms, the digit pad on each lever arm extending outward of the cap.

9. The needle assembly of claim 4, wherein the closed end portion comprises a first cylinder and the open end portion comprises a second cylinder, and wherein the second cylinder has a larger diameter that the first cylinder sized to affixed on the hub of the needle assembly.

10. The needle assembly of claim 1, wherein each lever arm comprises an angled distal edge.

11. The needle assembly of claim 1, wherein each lever arm comprises an arcuate angled distal edge.

12. The needle assembly of claim 1, wherein each lever arm forms a radially outwardly extending paddle.

13. The needle assembly of claim 12, wherein the paddle includes a digit pad on opposing surfaces of the paddle.

14. A needle assembly, comprising:
a needle cannula having a proximal end and a distal end with a puncture tip;
a hub having a proximal end and a distal end, the hub supporting the needle cannula at a distal end, a twist fitting connector provided at the proximal end; and
a radially outwardly extending lever arm having a digit pad configured on the hub in an anatomically preferential position when the needle is affixed to a syringe with the connector such that a user is induced to palm-grasp a barrel of the syringe in a manner that points the puncture tip away from a user when receiving a digit on the digit pad during mating and demating from the syringe.

15. The needle assembly of claim 10, wherein the radially outwardly extending lever arm is a radially outwardly extending paddle having a digit pad configured on the hub in an anatomically preferential position when the needle is affixed to a syringe.

16. The needle assembly of claim 15, wherein a pair of the radially outwardly extending lever arms are provided extending in opposition from the hub.

17. The needle assembly of claim 16, further comprising a cap having a closed distal end for receiving and protecting the puncture tip of the needle, a pair of diametrically opposed axial slots contiguous with an open leading end portion of the cap each configured to received one of the paddles, the digit pad on each paddle extending outward of the cap.

18. The needle assembly of claim 12, wherein the closed end portion comprises a first cylinder and the open end portion comprises a second cylinder, and wherein the second cylinder has a larger diameter that the first cylinder sized to affixed onto the hub of the needle assembly.

19. The needle assembly of claim 11, wherein one of the at least one of the lever arms has a visual and/or tactile feedback indicia on one of a removal surface of the digit pad and a mounting surface of the digit pad.

20. A method of demating a needle from a syringe, comprising:
providing a needle assembly having a cannula, a hub, a twist fitting connector on the hub configured to affix the hub onto a syringe, and at least one radially outwardly extending lever arm on the hub having a digit pad affixed onto a syringe; and
while affixed onto the syringe, pointing the cannula away from a user;
engaging a user's digit with the digit pad; and
pivotally urging the digit pad with the user's digit to rotate and decouple the twist fitting connector and remove the needle assembly from the syringe.

21. The method of claim 20, further comprising providing an indicia visually and/or tactilely perceptible to a user to indicate the digit pad, when urged that decouples the needle assembly from the syringe.

22. The method of claim 20, further comprising providing a pair of radially outwardly extending lever arms extending from the hub in opposed relation and engaging a user's digit with one finger pad from one of the pair of lever arms.

23. The method of claim 22, wherein the twist fitting connector is a luer lock connector and pivotally urging comprises unthreading the luer lock connector.

24. The method of claim 20, wherein prior to engaging a user's digit with the digit pad, approaching the digit pad from proximate the syringe moving toward the cannula.
